(19) United States
(12) Patent Application Publication
(10) Pub. No.: US 2025/0034064 A1
(43) Pub. Date: Jan. 30, 2025

(54) SYNTHESIS, PROCESS DEVELOPMENT AND OPTIMIZATION OF HYDROGENATED ALPHA METHYL STYRENE DIMER

(71) Applicant: VGP IPCO LLC, Lexington, KY (US)

(72) Inventors: Rajkumar Rajule, Kalyan West (IN); Keshav Sopan Badhe, Mumbai (IN); Ning Ren, Naperville, IL (US)

(21) Appl. No.: 18/752,951

(22) Filed: Jun. 25, 2024

Related U.S. Application Data
(60) Provisional application No. 63/515,339, filed on Jul. 24, 2023.

(51) Int. Cl. C07C 5/10

(52) U.S. Cl. CPC C07C 5/10; C07C 2523/755; C07C 2523/76

(57) ABSTRACT

A procedure for hydrogenation of alpha methyl styrene dimer with a Nickel on silica catalysts that is scalable, economical, and safe is provided.

### REFERENCE TO RELATED APPLICATIONS

This application claims priority from U.S. Provisional Patent Application No. 63/515,339, filed on Jul. 24, 2023, which is incorporated by reference herein in its entirety.

### BACKGROUND

The global demand for improved fuel economy and reduced emissions has led to significant improvement in powertrain efficiency, one of the improvements is continuous variable transmissions (CVT), which requires efficient traction fluid which can operate at low temperature and has a good traction coefficient. Studies of traction coefficient is well documented and its function of molecular structure. The commercially well-known fluid “Hydrogenated alpha methyl styrene dimer” (HAD) has a good traction coefficient and has an adequate low temperature property. Earlier we have developed scalable synthesis of HAD using Ru/C and Rh/C. The process developed is efficient and effective, but as commodities prices have risen, so does the cost of catalyst. It was imperative to find new catalyst which are low cost and effective for the synthesis of HAD.

Most known chemical processes use Raney Nickel as catalyst, and reaction is carried out at very high temperature and pressure. Nickel is economical to use, but with poor recyclability and have safety issues while handling at larger scale.

Therefore, there is a need in the field to have a safe, green and economical process on bulk scale, with less loading of the catalyst and better yields.

### SUMMARY

The present disclosure provides hydrogenation of alpha methyl styrene dimer with a Nickel on silica catalysts that is scalable, economical, and safe

The method of producing hydrogenated compounds such a hydrogenated alpha methyl styrene dimer compound includes: adding a Nickel on silica catalyst and compound to be hydrogenated to form a catalyst and compound reaction mixture and heating the reaction mixture under pressure until hydrogenation of the compound is complete followed by filtering the reaction mixture to obtain a hydrogenated compound. Importantly, the method yields more than 94% hydrogenated compound when referenced against starting material.

The time, pressure, temperature, amounts of catalyst and starting material may all be varied. The catalyst of the reaction may be recovered after reaction completion and can be used in the next batch with additional topping of fresh catalyst.

### BRIEF DESCRIPTION OF THE DRAWINGS

In the accompanying figures, chemical formulas, chemical structures, and experimental data are given that, together with the detailed description provided below, describe example embodiments of the claimed invention.

FIG. 1A-1B shows the NMR spectra (1A) and tabular results (1B) for a sample according to an aspect of the invention.

FIG. 2A-2B shows the NMR spectra (2A) and tabular results (2B) for a sample according to an aspect of the invention.

FIG. 3A-3B shows the NMR spectra (3A) and tabular results (3B) for a sample according to an aspect of the invention.

FIG. 4A-4B shows the NMR spectra (4A) and tabular results (4B) for a sample according to an aspect of the invention.

FIG. 5A-5B shows the NMR spectra (5A) and tabular results (5B) for a sample according to an aspect of the invention.

### DETAILED DESCRIPTION

A method for preparing hydrogenated compounds including a first step of adding to a reactor under nitrogen a Nickel on silica catalyst and adding a compound to be hydrogenated to the reactor thereby forming a catalyst and compound reaction mixture is now fully described. As a second step may include heating the reaction mixture under pressure until hydrogenation of the compound is complete followed by filtering the reaction mixture through a celite bed under nitrogen thereby obtaining a hydrogenated compound. Importantly, the method yields more than 94% hydrogenated compound when referenced against starting material.

The method as described herein is effective for hydrogenation of numerous compounds. The term compound referring to any chemical structure in need to hydrogenation. In fact the term compound may refer to a mixture of individual chemical structures in need of hydrogenation. In some aspects, the compound to be hydrogenated is a alpha methyl styrene dimer. In some aspects, the compound to be hydrogenated is a blend of alpha methyl styrene dimer and alpha methyl styrene dimer isomer. In some aspects, the compound to be hydrogenated is a 1:1 blend of alpha methyl styrene dimer and alpha methyl styrene dimer isomer.

In some aspects, the reaction mixture of the compound and the catalyst is heated to a temperature of between 50° C. and 165° C. until hydrogenation of the compound is complete. It is understood the temperature range can be varied according to the needs of the user and still be encompassed by the invention and may include for example, a range between 50° C. and 160° C., 50° C. and 155° C., 50° C. and 150° C., 55° C. and 165° C., 55° C. and 160° C., 55° C. and 155° C., 55° C. and 150° C., as well as any individual temperature found within this range such as 148° C. or any subset of ranges encompasses by 50° C. and 165° C. such as 57° C. and 142° C., for example.

According to some aspects, the hydrogenation method is characterized in that reaction mixture is maintained under a pressure of 10 Kg/cm² and 15 Kg/cm² until hydrogenation of the compound is complete. It is understood the pressure range can be varied according to the needs of the user and still be encompassed by the invention and may include for example, a pressure of between 10 Kg/cm² and 14 Kg/cm²′ a pressure of 10 Kg/cm² and 13 Kg/cm², a pressure of 11 Kg/cm² and 15 Kg/cm², a pressure of 11 Kg/cm² and 14 Kg/cm², a pressure of 11 Kg/cm² and 13 Kg/cm², a pressure of 12 Kg/cm² and 15 Kg/cm², a pressure of 12 Kg/cm² and 14 Kg/cm², as well as any individual pressure found within this range such as a pressure of 13.5 Kg/cm² or any subset of ranges encompasses by a pressure of 10 Kg/cm² and 15 Kg/cm² a pressure of between 13 Kg/cm² and 14 Kg/cm² for example.

According to some aspects, the hydrogenation method is completed in a reaction time of between 1.5 hours and 11 hours. It is understood the reaction time range can be varied according to the needs of the user and still be encompassed by the invention and may include for example, the reaction time may be between 1.5 hours and 10 hours, 1.5 hours and 9 hours, 1.5 hours and 8 hours, 1.5 hours and 7 hours, 2 hours and 11 hours, 2.5 hours and 11 hours, 3 hours and 11 hours, 3.5 hours and 11 hours, 4 hours and 11 hours. Further, the reaction time may be any individual time found within this range such as a reaction time of 7.2 hours or any subset of ranges encompassed by a reaction time of between 1.5 hours and 11 hours such as a reaction time of between 5.5 and 7 hours, for example.

According to some aspects, the hydrogenation method is performed in the presence of a catalyst that is Nickel on silica. In some aspects, the Nickel on silica catalyst is present at a loading weight % of between 0.5 wt % and 25 wt %. The loading weight % is calculated with respect to other reactants present in the reaction mixture. In some aspects the Nickel on silica catalyst is present at a loading weight % of between 1 wt % and 25 wt %, between 0.5 wt % and 25 wt %, between 1.5 wt % and 25 wt %, between 2 wt % and 25 wt %, between 3 wt % and 25 wt %, between 4 wt % and 25 wt %, between 5 wt % and 25 wt %, between 6 wt % and 25 wt %, between 7 wt % and 25 wt %, between 8 wt % and 25 wt %, between 9 wt % and 25 wt %, between 10 wt % and 25 wt %, between 0.5 wt % and 20 wt %, between 0.5 wt % and 15 wt %, between 0.5 wt % and 10 wt %, between 0.5 wt % and 5 wt %. Further, the Nickel on silica catalyst is present at a loading weight (wt) % of any individual wt % found within this range such as a 1.2 wt % or any subset of ranges encompassed by a loading weight % between loading weight % of between 0.5 wt % and 25 wt % such as a loading weight % of between 0.5 wt % and 1.5 wt %, for example.

According to some aspects, the hydrogenation method is performed in the presence of a catalyst that is solely consisting of Nickel on silica. That is Nickel is the only metal present in the catalyst and the catalyst lacks any other metal. The catalyst may be free of Pd, Ru, Rh, alumina, carbon. That is, according to many aspects of the invention, the amount of Pd, Ru, Rh, alumina or carbon in the catalyst is 0%.

The Nickel on silica catalyst further may be a about 40% to about 65% Nickel on silica catalyst, or 40% to 65% Nickel on silica catalyst. The Nickel on silica catalyst is present at any individual % found within this range such as a 60% or any subset of ranges encompassed by 0% to 65% Nickel on silica between 45% and 60%, for example.

These procedures result in routinely greater than a 94% yield and require no purification step. The yield may also be greater than 95%, or greater than 96%, or greater than 97% or greater than 98% or greater than 99% or greater than 99.9% or include a range of between or greater than 94% and greater than 99% or any range between 94% and 99%. The yield is calculated with reference to the amount of starting material. The yield may be represented by any single number found within the range of 94% yield to 99% yield, e.g. 95.7% and may also be encompassed by any range fully encompassed by the range of 94% yield to 99% yield, for example 95%-98%.

### EXAMPLES

### Example 1: Screening of the Catalyst for the Hydrogenation of Alpha Methyl Styrene Dimer to Hydrogenated Alpha Methyl Styrene Dimer

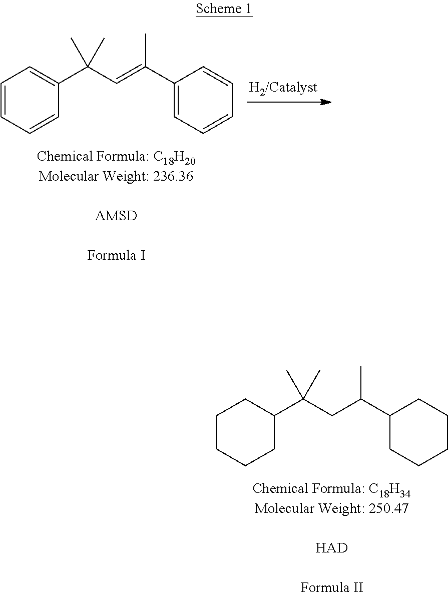

A screening of the catalyst for the hydrogenation of alpha methyl styrene dimer to hydrogenated alpha methyl styrene dimer given in Table 1.

Most literature mentions hydrogenation using Raney Nickel, which requires very high temperature and pressure, to scaling up on larger scale is not safe. Raney Nickel and Pd(OH)2/C did not work at low temperature and pressure. So herein we have screened Ru/Alumina, Pd/Alumina, Nickel/Si/Alumina, Pd/C and Ni on silica. Out of all these catalysts we found Nickel on silica most efficient and cost effective.

TABLE 1
-AMSD Hydrogenation

| | AMS | Catalyst | | | Reaction | | |
|---|---|---|---|---|---|---|---|
| Exp. | dimer | (Loading | Temp. | Pressure | time | Yield | |
| No. | (g) | wt. %) | (° C.) | (Kg/cm2) | (h) | (g) | Remarks |
| 1. | 200 | Raney | 60- | 35 | 4 | | Safety issue |
| | | Nickel | 180- | | | | |
| | | (8.5%) | 200° C. | | | | |

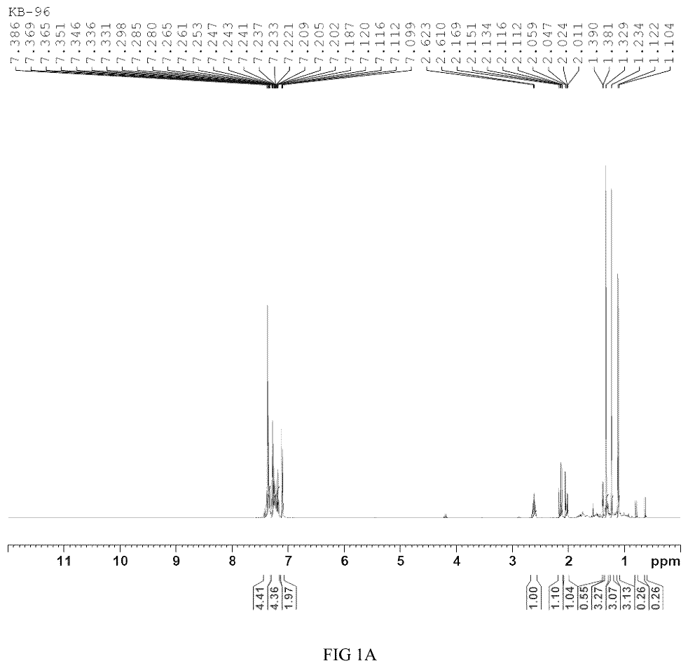

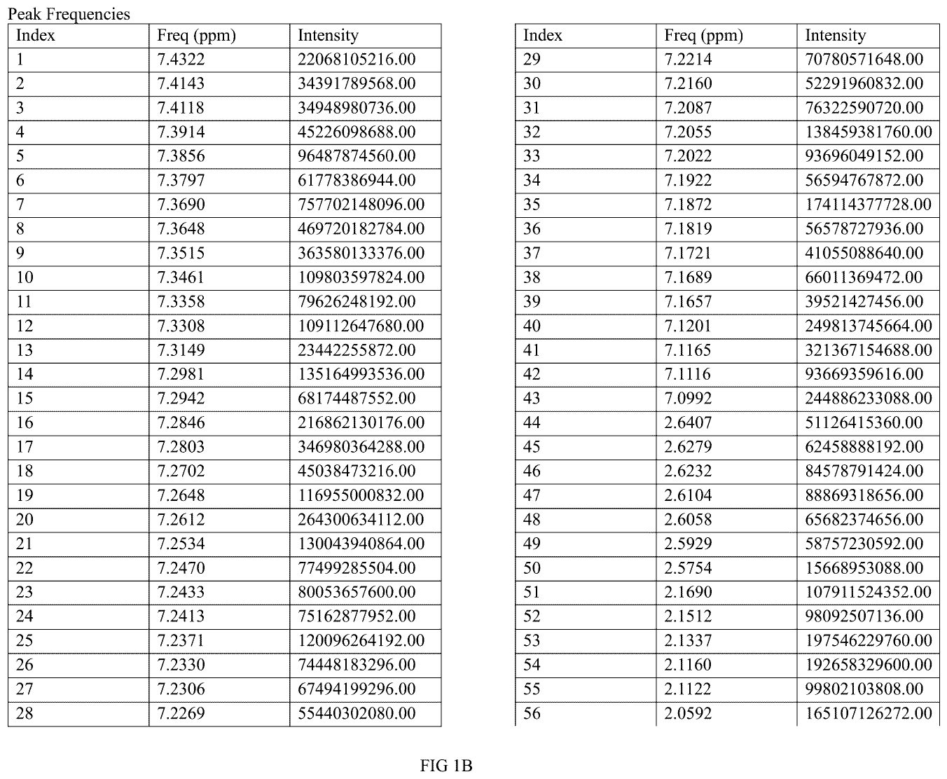

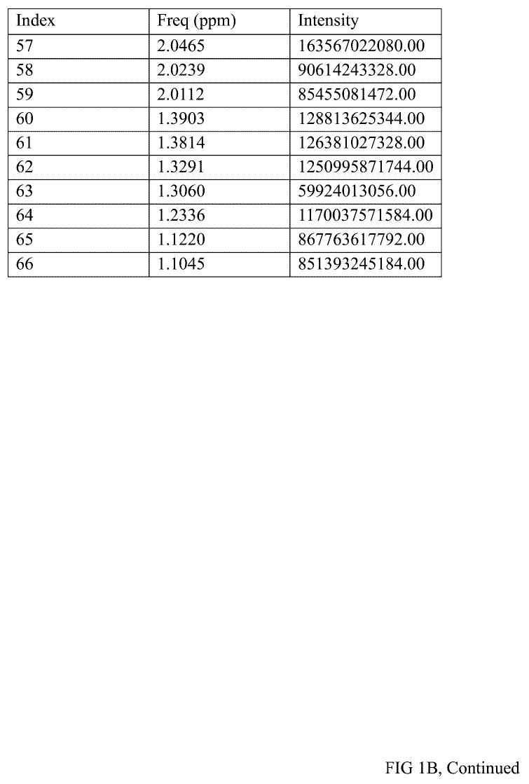

AMSD Hydrogenation—Screening of catalysts

| | AMS | Catalyst | | | Reaction | | | |
|---|---|---|---|---|---|---|---|---|
| Exp. | dimer | (Loading | Temp. | Pressure | time | Yield | | |
| No. | (g) | wt. %) | (° C.) | (Kg/cm2) | (h) | (g) | Remarks | NMR |
| 1. | 200 | 5% | 30- | 13-14 | 2 | 206 | NMR shows | |
| | | Ru/Alumina | 160° C. | | | (97%) | absence of | |
| | | (5%) | | | | | aromatic peaks | |
| 2. | 200 | 5% | 46- | 13-14 | 5 | 207 | TLC indicate | |
| | | Ru/Alumina | 160° C. | | | (98%) | reaction is | |
| | | (1%) | | | | | complete | |
| 3. | 200 | 5% | 32- | 13-14 | 7.5 | 205 | NMR shows | |
| | | Pd/Alumina | 160° C. | | | (97%) | absence of | |
| | | (5%) | | | | | aromatic peaks | |
| 4. | 200 | Nickel/ | 55- | 13-14 | 7 | 202 | NMR shows | FIG. 1 |
| | | Silica & | 150° C. | | | (95%) | presence of | |
| | | alumina | | | | | aromatic peaks | |
| | | (20%) | | | | | | |
| 5. | 200 | Nickel/ | 55- | 13-14 | 3.5 | 204- | NMR shows | |
| | | Silica | 150° C. | | | 205 | absence of | |
| | | (20%) | | | | (95%) | aromatic peaks | |
| 6. | 200 | 5% Pd/C | 60- | 13-14 | 10.5 | 204 | TLC indicate | |
| | | (1%) | 160° C. | | | (96%) | reaction is | |
| | | | | | | | complete | |

### Example 2: Optimization of Nickel on Silica

Optimization of Nickel on silica with respect to its loading and found 1% loading most efficient as shown in Table 2.

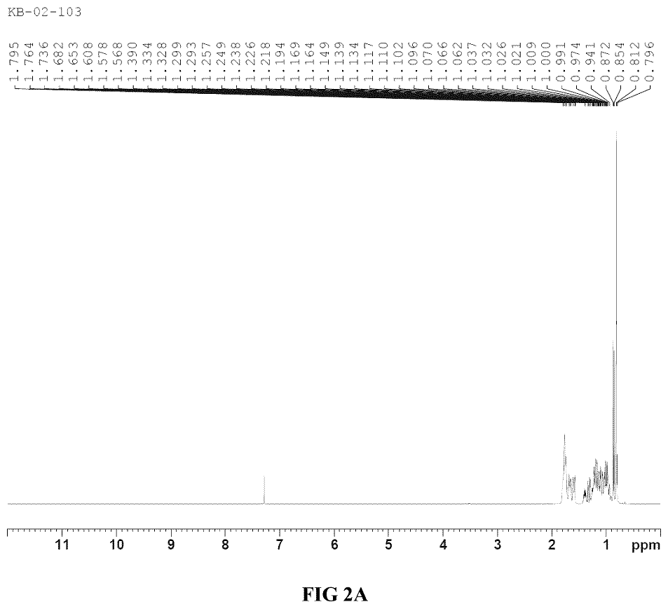

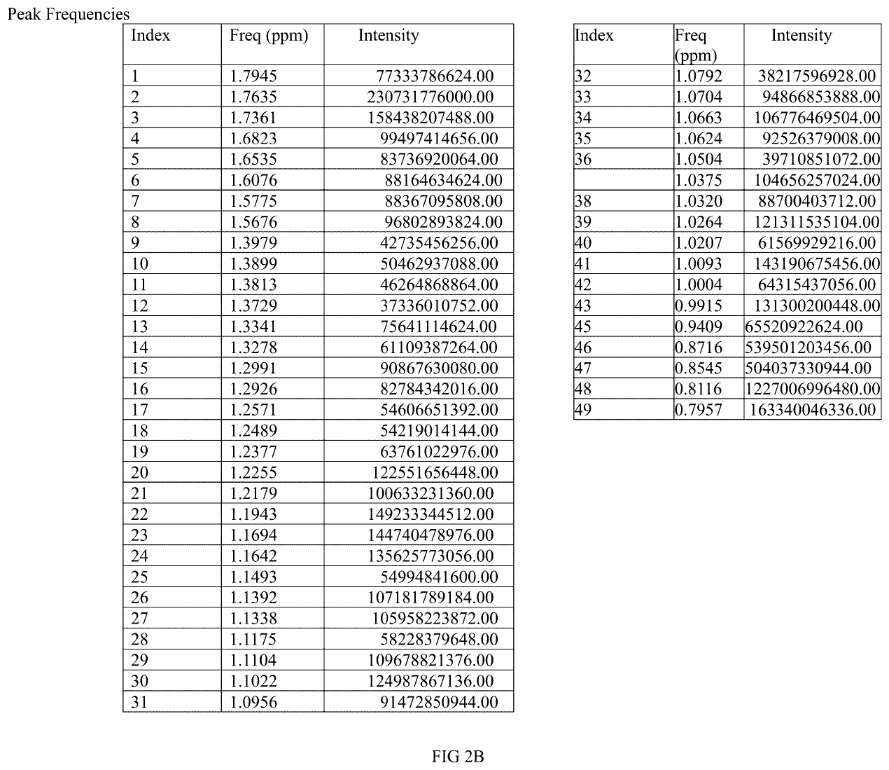

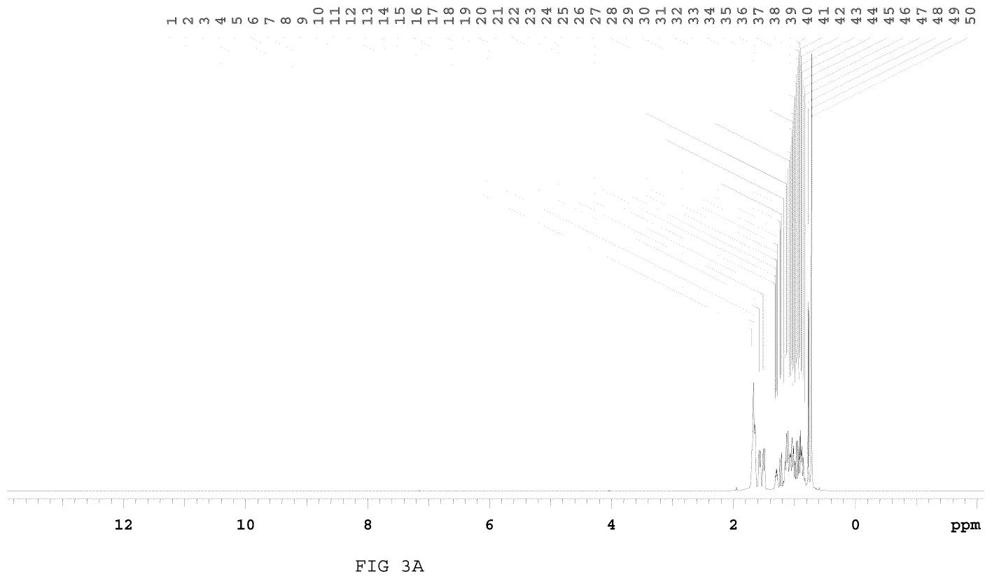

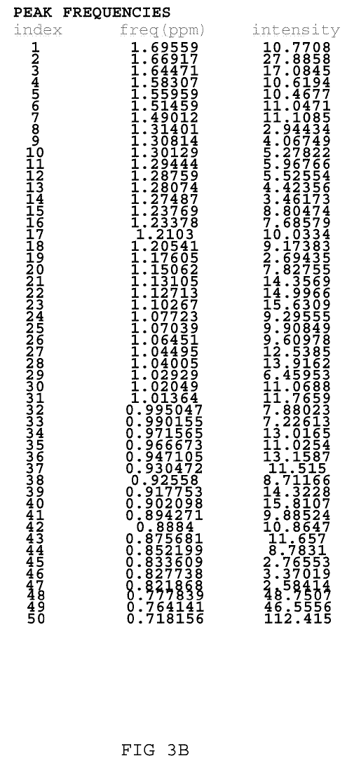

TABLE 2

| | AMS | Catalyst | | | Reaction | | | |
|---|---|---|---|---|---|---|---|---|
| Exp. | dimer | (Loading ) | Temp. | Pressure | time | Yield | | |
| No. | (g) | wt. % | (° C.) | (Kg/cm2) | (h) | (g) | Remarks | NMR |
| 1. | 200 | Nickel/Silica | 55- | 13-14 | 3.5 | 200 | NMR shows | FIG. 2 |
| | | (20%) | 150° C. | | | (94%) | absence of | |
| | | | | | | | aromatic peaks | |
| 2. | 200 | Nickel/Silica | 55- | 13-14 | 4 | 211 | NMR shows | |
| | | (15%) | 150° C. | | | (99%) | absence of | |
| | | | | | | | aromatic peaks | |
| 3. | 200 | Nickel/Silica | 55- | 13-14 | 4.5 | 209 | NMR shows | |
| | | (5%) | 150° C. | | | (99%) | absence of | |
| | | | | | | | aromatic peaks | |
| 4. | 200 | Nickel/Silica | 55- | 13-14 | 6 | 208 | NMR shows | |
| | | (2.5%) | 160° C. | | | (98%) | absence of | |
| | | | | | | | aromatic peaks | |
| 5. | 200 | Nickel/Silica | 55- | 13-14 | 6.5 | 208 | NMR shows | FIG. 3 |
| | | (1%) | 160° C. | | | (98%) | absence of | |
| | | | | | | | aromatic peaks | |

### Example 3: Co-Hydrogenation of a Mixture of Alpha Methyl Styrene Dimer and its Isomer

The result obtained from the above studies, implemented on mixture of alpha methyl styrene dimer and its isomer in the ratio of 1:1 given in Table 3.

The catalyst Ni on silica with 1% loading gave the desired mixture, with good yield.

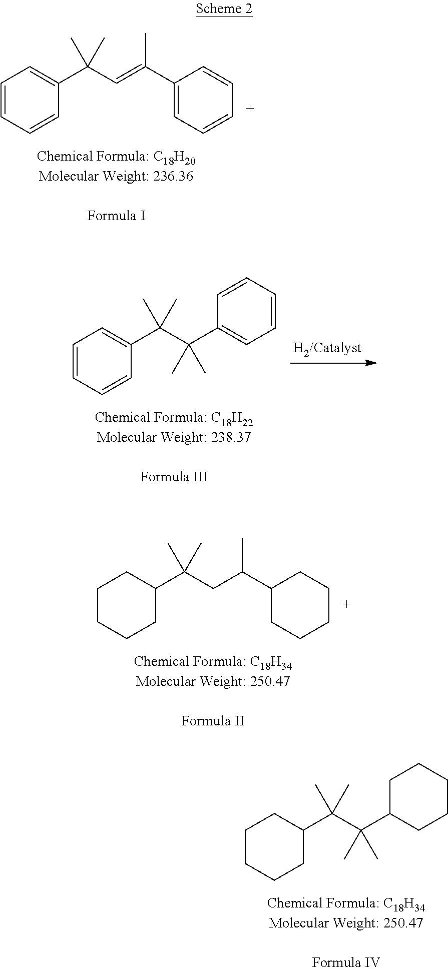

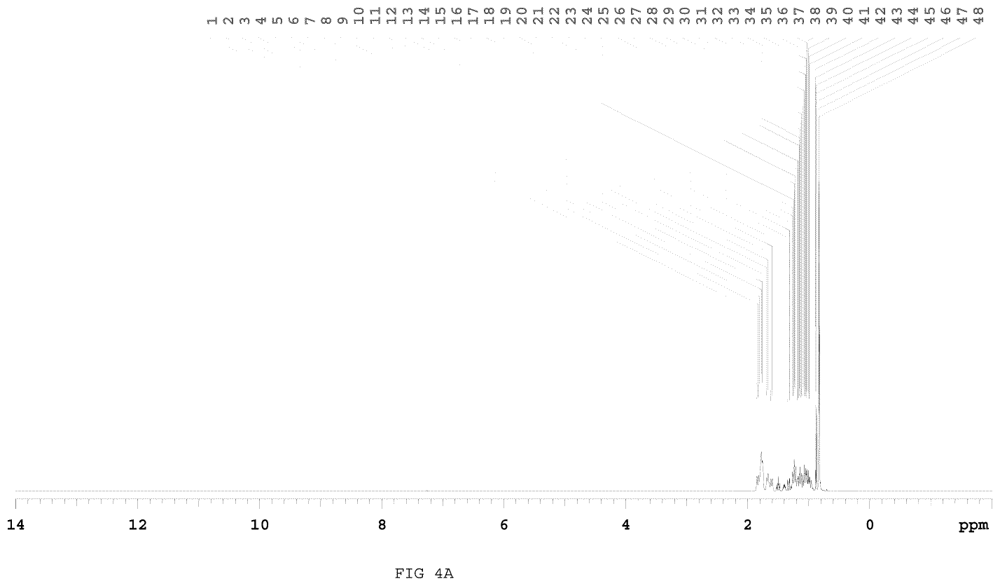

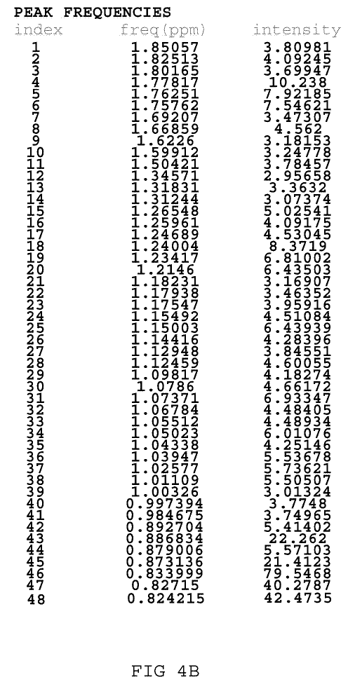

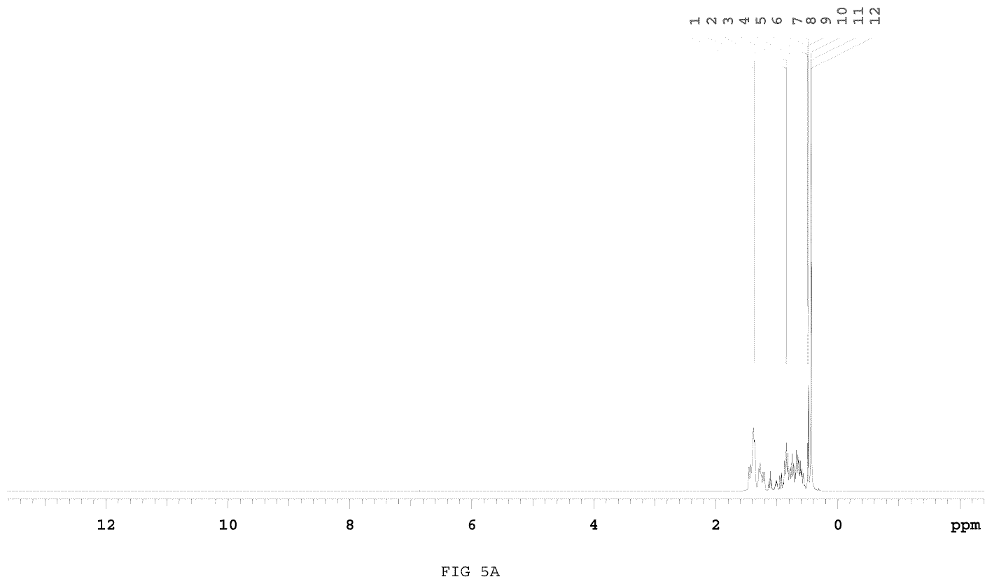

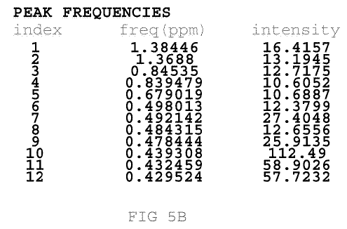

TABLE 3
AMSD and Iso-HAD Hydrogenation

| | AMS | | | | | | | |
|---|---|---|---|---|---|---|---|---|
| | dimer + | | | | | | | |
| | Iso-HAD | Catalyst | | | Reaction | | | |
| Exp. | (1:1) | (Loading | Temp. | Pressure | time | Yield | | |
| No. | (g) | wt. %) | (° C.) | (Kg/cm2) | (h) | (g) | Remarks | NMR |
| 1. | 200 | Nickel/ | 57- | 13-14 | 3.5 | 210 | NMR shows | FIG. 4 |
| | | Silica | 160° C. | | | (99%) | absence of | |
| | | (5%) | | | | | aromatic peaks | |
| 2. | 200 | Nickel/ | 65- | 13-14 | 6 | 207 | NMR shows | FIG. 5 |
| | | Silica | 160° C. | | | (98%) | absence of | |
| | | (2%) | | | | | aromatic peaks | |
| 3. | 200 | Nickel/ | 70- | 13-14 | 9 | 208 | NMR shows | |
| | | Silica | 160° C. | | | (98%) | absence of | |
| | | (1%) | | | | | aromatic peaks | |

Certain embodiments have been described in the form of examples. It is impossible to depict every potential application. Thus, while the embodiments are described in considerable detail, it is not the intention to restrict or in any way limit the scope of the appended claims to such detail, or to any particular embodiment.

To the extent that the term “includes” or “including” is used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A or B or both.” When “only A or B but not both” is intended, then the term “only A or B but not both” will be employed. Thus, use of the term “or” herein is the inclusive, and not the exclusive use. As used in the specification and the claims, the singular forms “a,” “an,” and “the” include the plural. Finally, where the term “about” is used in conjunction with a number, it is intended to include ±10% of the number. For example, “about 10” may mean from 9 to 11. The term HAD may be used to refer to a hydrogenated alpha methyl styrene dimer or hydrogenated dimers of alpha olefins, or any other term referring to the figure shown in Formula I or defined as HAD.

As stated above, while the present application has been illustrated by the description of embodiments, and while the embodiments have been described in considerable detail, it is not the intention to restrict or in any way limit the scope of the appended claims to such detail. Additional advantages and modifications will readily appear to those skilled in the art, having the benefit of this application. Therefore, the application, in its broader aspects, is not limited to the specific details and illustrative examples shown. Departures may be made from such details and examples without departing from the spirit or scope of the general inventive concept.

1. A method for preparing hydrogenated compound comprising:
adding to a reactor under nitrogen a Nickel on silica catalyst;
adding a compound to be hydrogenated to the reactor thereby forming a catalyst and compound reaction mixture;
heating the reaction mixture under pressure until hydrogenation of the compound is complete; and
filtering the reaction mixture through a celite bed under nitrogen thereby obtaining a hydrogenated compound,
wherein the method yields 94% hydrogenated compound.

2. The method of claim 1, wherein the compound to be hydrogenated is a alpha methyl styrene dimer, or a blend of alpha methyl styrene dimer and alpha methyl styrene dimer isomer.

3. The method of claim 1, wherein the reaction mixture is heated to a temperature of between 50° C. and 165° C. until hydrogenation of the compound is complete.

4. The method of claim 1, wherein the reaction mixture is maintained under a pressure of 10 Kg/cm² and 15 Kg/cm² until hydrogenation of the compound is complete.

5. The method of claim 1, wherein the hydrogenation of the compound is completed in a reaction time of between 1.5 hours and 11 hours.

6. The method of claim 1, wherein the Nickel on silica is present at a loading weight % of between 0.5 wt % and 25 wt %.

7. The method of claim 1, wherein the Nickel on silica catalyst is 20-80% Nickel on silica.
